(19) United States
(12) Patent Application Publication
(10) Pub. No.: US 2020/0129411 A1
(43) Pub. Date: Apr. 30, 2020

(54) Solid Bar Shampoo

(71) Applicant: Lonza Ltd., Visp (CH)

(72) Inventors: Adam Griffiths, Great Sutton (GB); Louise Reay, Bebington (GB)

(21) Appl. No.: 16/665,344

(22) Filed: Oct. 28, 2019

Related U.S. Application Data
(60) Provisional application No. 62/752,122, filed on Oct. 29, 2018.

(51) Int. Cl. A61K 8/86; A61K 8/02; A61Q 5/02; A61K 8/37; A61K 8/46; A61K 8/44; A61K 9/00; A61K 31/4412; A61K 31/44; A61K 31/444; A61K 31/496; A61K 31/4164; A61K 33/04

(52) U.S. Cl. CPC A61K 8/86; A61K 2800/30; A61Q 5/02; A61K 8/375; A61K 8/466; A61K 8/442; A61K 8/463; A61K 9/0014; A61K 31/4412; A61K 31/44; A61K 31/444; A61K 31/496; A61K 31/4164; A61K 33/04; A61K 2800/596; A61K 8/0229

(57) ABSTRACT

The present invention provides a shampoo bar composition, at least 60 w/w % of one or more surfactants, wherein at least one or more of the surfactant comprises a polyglyceryl ester. The solid bar composition contains lower levels of anionic surfactants and may further be free of sulfate or sulfonate surfactants.

### RELATED APPLICATIONS

The present application is based on and claims priority to U.S. Provisional Patent application Ser. No. 62/752,122, filed on Oct. 29, 2018, which is incorporated herein by reference.

### FIELD OF INVENTION

The disclosure relates to a solid bar shampoo composition containing polyglyceryl esters.

### BACKGROUND OF THE INVENTION

Most commercial shampoos are in liquid form, made from soaps or mixtures of soaps. These shampoos are often packaged in containers such as plastic bottles or sachets. Such containers can result in leakage occurring, failure of lids and high waste generated from packaging and from use of higher volumes of shampoo during application, in comparison to shampoo bars. The use of liquid shampoos is also expensive and environmentally unfriendly due to the transport of large volumes of liquid.

Solid shampoo bars are also known. The current commercial solid shampoo bars have high anionic surfactant levels and/or contain sulfate and sulfonate based surfactants. High anionic surfactant-based shampoos are known to be degreasing and may feel harsh on the skin or hair; and are known to strip hair of color.

There is a need in the art to provide a solid bar shampoo containing less anionic surfactants without affecting the solid form of the shampoo and simultaneously having sufficient lathering and cleansing actions for both skin and hair. Reducing the levels of anionic surfactants present in such compositions is desirable economically and environmentally friendly. The present invention provides an answer to that need.

### SUMMARY OF THE INVENTION

In one aspect, the present invention provides a solid bar shampoo composition containing at least 60 w/w % of one or more surfactants based on the total weight of the composition; wherein the at least one or more surfactant comprises a polyglyceryl ester.

In one embodiment, in addition to the at least one surfactant, the solid bar shampoo further contains at least one or more of an anionic surfactant, cationic surfactant, nonionic surfactant or amphoteric surfactant.

In another embodiment, the composition comprises 60 w/w %-99 w/w % of one or more surfactants, based on the total weight of the composition; wherein at least one of the surfactants contains a polyglyceryl ester, and at least one additional surfactant is an anionic surfactant, a cationic surfactant, a nonionic surfactant or an amphoteric surfactant.

In one particular embodiment, the polyglyceryl ester contains one or more of a polyglyceryl-10 laurate, polyglyceryl-10 decaoleate; polyglyceryl-3 monostearate; polyglyceryl-6 distearate, polyglyceryl-10 stearate; polyglyceryl-10 oleate; polyglyceryl-10 dipalmitate, or polyglyceryl-10 caprylate/caprate.

In one particular embodiment, the anionic surfactant contains one or more of a sodium lauroyl methyl isethionate, sodium cocoyl methyl isethionate, sodium oleoyl methyl isethionate, olefin sulphonates, sodium lauryl sulfate, olefin sulphonate, disodium laureth sulfosuccinate, sodium lauryl sulfoacetate, sulfonated methyl esters, sodium cocoyl glutamate, or sodium lauroyl sarcosinate.

In another embodiment, the amphoteric surfactant contains disodium cocoamphodiacetate.

In yet another embodiment, the nonionic surfactant contains glyceryl stearate (and) peg-100 stearate.

In one aspect of the present invention, the composition is essentially free of sulfate based surfactants.

In another aspect of the present invention, the composition is a non-saponified bar shampoo.

In one embodiment, the present composition further contains one or more ingredients selected from fragrances, dyes, structuring aids, fillers, pH adjustment agents, chelating agents, antidandruff agents, biocidal agent, conditioning agents, essential oils, binding agent, natural extracts, botanical extracts, organic acids, alcohols, emulsifiers, natural plant material, fats, oils, essential fatty acids, preservatives, antioxidants, moisturizing agents, or coloring agents.

In one particular embodiment, the present invention contains 1 w/w %-80 w/w % of one or more polyglyceryl esters, up to 80 w/w % of one or more anionic surfactants, 1 w/w %-25 w/w % of one or more amphoteric surfactants, 1 w/w %-50 w/w % of one or more nonionic surfactants, 0.01 w/w %-25 w/w % of one or more cationic surfactants, 0.01 w/w %-20 w/w % essential oils, 0.1 w/w %-10 w/w % fatty alcohol, 0.1 w/w %-10 w/w % fatty acid, 0.01 w/w %-5 w/w % fragrances, or 0.01 w/w %-5 w/w % dyes.

In yet another embodiment, a solid bar shampoo composition is described, containing (a) at least 60% of one or more surfactants, wherein the one or more surfactant comprises a polyglyceryl ester and (b) at least one or more antidandruff agent.

In one embodiment, the antidandruff agent contains at least one or more of hydroxypyridinones or derivatives thereof, octopirox, pyrithione derivatives or salts thereof, bispyrithione, octenidine or salts thereof, ketoconazole, climbazole, selenium or salts thereof, salicylic acid or salts thereof, or tea tree oil.

In a further embodiment, the present invention contains 1 w/w %-80 w/w % of one or more polyglyceryl esters, up to 80 w/w % of one or more anionic surfactants, 1 w/w %-25 w/w % of one or more amphoteric surfactants, 1 w/w %-50 w/w % of one or more nonionic surfactants, 0.01 w/w %-25 w/w % of one or more cationic surfactants, 0.01 w/w %-20 w/w % essential oils, 0.1 w/w %-10 w/w % fatty alcohol, 0.1 w/w %-10 w/w % fatty acid, 0.01 w/w %-10 w/w % antidandruff agent, 0.01 w/w %-5 w/w % fragrances, 0.01 w/w %-2 w/w % moisturizing agent, or 0.01 w/w %-5 w/w % dyes.

These and other aspects will become apparent when reading the detailed description of the invention.

### DETAILED DESCRIPTION

It has now been surprisingly found that a solid bar shampoo composition as described containing polyglyceryl esters as at least one surfactant wherein such compositions contain low anionic surfactant levels and may be prepared essentially free of sulfate and/or sulfonate based surfactants.

The term “solid” herein refers a composition in the solid state under normal atmospheric conditions (i.e. at a pressure of 1 atmosphere and 298 K).

The term “essentially free” herein, refers to a composition containing less than 1 w/w % of a sulfate or sulfonate based surfactants.

It has now been surprisingly found that the addition of polyglyercyl esters as at least one surfactant may be added to a solid bar shampoo composition while maintaining the physical and beneficial properties of solid shampoos. The addition of at least one polyglyceryl ester, allows for the reduction or elimination of anionic surfactants, thereby overcoming current issues with solid bar shampoos having high anionic surfactants. The solid bar shampoo composition, containing polyglyceryl esters as at least one surfactant, maintains the lathering properties, rigidity of the bar shampoo having a moderate hardness, smooth bar appearance, exhibits controlled water stability, good foaming properties, and good rinsability, whilst providing conditioning properties to the skin and hair.

The present disclosure provides an answer to that need of having a solid bar shampoo, having low in anionic surfactant content by containing polyglyceryl esters as at least one of the surfactants used within the composition. The addition of polyglyceryl esters reduces the amount of anionic surfactants needed, while maintaining the physical and cleansing properties of the solid bar shampoo as described. The present invention therefore provides a solid bar shampoo composition, wherein the surfactant contains one or more polyglyercyl esters and wherein the composition is a non-saponified bar shampoo. Additionally, the solid bar shampoo contains decreased amounts of anionic surfactants and may be essentially free of sulfate and/or sulfonate based surfactants; thereby overcoming the difficulties of sulfate-based and liquid shampoos. Additionally, the use of polygylceryl esters within the solid bar shampoo composition maintains its lathering, foaming, solid form and cleaning properties, without the need of a high anionic surfactants composition.

One aspect of the present invention provides a shampoo bar composition comprising at least 60 w/w % of one or more surfactants, wherein at least one or more surfactant comprises a polyglyceryl ester, based on the total weight of the composition.

Typically the present invention comprises at least one or more surfactants wherein at least one or more comprises anionic surfactants, cationic surfactants, nonionic surfactants or amphoteric surfactants; wherein at least one of the surfactants contains a polyglyceryl ester, and at least one additional surfactant is an anionic surfactant, a cationic surfactant, a nonionic surfactant or an amphoteric surfactant.

Alternately, the present invention provides a solid bar shampoo composition whereby the composition is a non-saponified bar shampoo.

Suitably, the solid bar shampoo composition comprises 60 w/w %-99 w/w % of one or more surfactants based on the total weight of the composition, wherein at least one of the surfactants comprises a polyglyceryl ester, and may contain, in addition to the polyglyceryl ester, at least one or more anionic surfactants, cationic surfactants, nonionic surfactants or amphoteric surfactants.

Desirably, the solid bar shampoo composition comprises 70 w/w %-85 w/w % of one or more surfactants based on the total weight of the composition, wherein at least one of the surfactants comprises a polyglyceryl ester, and may contain, in addition to the polyglyceryl ester, at least one or more anionic surfactants, cationic surfactants, nonionic surfactants or amphoteric surfactants.

Polyglyceryl esters, useable as surfactants, in the present invention may be formed from saturated, unsaturated, natural or synthetic fatty acids, and the like. For instance, saturated fatty acids include caprylic acid, capric acid, lauric acid, myristic acid, palmitic acid, stearic acid, arachidic acid, behenic acid, lignoceric acid, cerotic acid, oleic acid, decaoleic acid, combinations thereof, derivatives thereof, and the like.

Furthermore, the polyglyceryl esters are derived from (a) a polyglycerol component built up from 2 to 12 molecules of glycerol, based on an average, and (b) a fatty acid comprises a caprylic acid, capric acid, lauric acid, myristic acid, palmitic acid, stearic acid, arachidic acid, behenic acid, lignoceric acid, cerotic acid, oleic acid, decaoleic acid, or mixtures thereof, and the like.

Examples of polyglyceryl esters, include, but are not limited to, polyglyceryl monodecaoleate such as polyglyceryl-10 decaoleate; polyglyceryl monooleate such as polyglyceryl-2-monooleate, polyglyceryl-3 monooleate, polyglyceryl-4 monooleate, polyglyceryl-6 monooleate, or polyglyceryl-10 monooleate; polyglyceryl dioleate such as polyglyceryl-2 dioleate, polyglyceryl-3 dioleate, polyglyceryl-5 dioleate, polyglyceryl-6 dioleate or polyglyceryl-10 dioleate; polyglyceryl trioleate such as polyglyceryl-5 trioleate or polyglyceryl-10 trioleate; polyglyceryl tetraoleate such as polyglyceryl-2 tetraoleate, polyglyceryl-6 tetraoleate, or polyglyceryl-10 tetraoleate; polyglyceryl pentaoleate such as polyglyceryl-4 pentaoleate, polyglyceryl-6 pentaoleate, or polyglyceryl-10 pentaoleate; polyglyceryl heptaoleate such as polyglyceryl-6 heptaoleate, polyglyceryl-10 heptaoleate; polyglyceryl monostearate such as polyglyceryl-2 monostearate, polyglyceryl-3 monostearate, polyglyceryl-4 monostearate, polyglyceryl-5 monostearate, polyglyceryl-6 monostearate or polyglyceryl-10 monostearate; polyglyceryl distearate such as polyglyceryl-2 distearate, polyglyceryl-3 distearate, polyglyceryl-4 distearate, polyglyceryl-6 distearate, or polyglyceryl-10 distearate; polyglyceryl tristearate such as polyglyceryl-4 tristearate, polyglyceryl-5 tristearate, polyglyceryl-6 tristearate, or polyglyceryl-10 tristearate; polyglyceryl tetrastearate such as polyglyceryl-2 tetrastearate; polyglyceryl pentastearate such as polyglyceryl-4 pentastearate, polyglyceryl-6 pentastearate, or polyglyceryl-10 pentastearate; polyglyceryl heptastearate such as polyglyceryl-10 heptastearate; polyglyceryl isostearate such as polyglyceryl-2 isostearate, polyglyceryl-3 isostearate, polyglyceryl-4 isostearate, polyglyceryl-6 isostearate, or polyglyceryl-10 isostearate; polyglyceryl diisostearate such as polyglyceryl-2 diisostearate. polyglyceryl-3 diisostearate, polyglyceryl-4 diisostearate, polyglyceryl-6 diisostearate, polyglyceryl-10 diisostearate, or polyglyceryl-15 diisostearate; polyglyceryl triisostearate such as polyglyceryl-2 triisostearate, polyglyceryl-3 triisostearate, polyglyceryl-5 triisostearate, polyglyceryl-10 triisostearate; polyglyceryl tetraisostearate such as polyglyceryl-2 tetraisostearate; polyglyceryl caprylate such as polyglyceryl-2 caprylate, polyglyceryl-3 caprylate, polyglyceryl-4 caprylate, polyglyceryl-6 caprylate, or polyglyceryl-10 caprylate; polyglyceryl dicaprylate such as polyglyceryl-5 dicaprylate; polyglyceryl sesquicaprylate such as polyglyceryl-2 sesquicapyrlate; polyglyceryl octacaprylate such as polyglyceryl-6 octacaprylate; polyglyceryl caprate such as polyglyceryl-2 caprate, polyglyceryl-3 caprate, polyglyceryl-4 caprate, polyglyceryl-5 caprate, polyglyceryl-6 caprate, polyglyceryl-10 caprate: polyglyceryl dicaprate such as polyglyceryl-3 dicaprate or polyglyceryl-6 dicaprate; polyglyceryl caprylate/caprate such as polyglyceryl-4 capyrlateicaprate, polyglyceryl-6 caprylate/caprate, or polyglyceryl-10 caprylate/caprate; polyglyceryl palmitate such as polyglyceryl-2 palmitate, polyglyceryl-3 palmitate, polyglyceryl-6 palmitate or polyglyceryl-10 palmitate; polyglyceryl dipalmitate such as polyglyceryl-6 dipalmitate or polyglyceryl-10 dipalmitate; polyglyceryl tetrabehenate such as polyglyceryl-6 tetrabehenate; polyglyceryl myristate such as polyglyceryl-6 myristate or polyglyceryl-10 myristate; polyglyceryl rincinoleate such polyglyceryl-6 polyricinoleate or polyglyceryl-10 ricinoleate; polyglyceryl-10 laurate, other derivatives thereof, or mixtures thereof, and the like.

Suitably, the polyglyceryl ester comprises one or more of a polyglyceryl-10 laurate, polyglyceryl-10 decaoleate; polyglyceryl-3 monostearate; polyglyceryl-6 distearate, polyglyceryl-10 stearate; polyglyceryl-10 oleate; polyglyceryl-10 dipalmitate, or polyglyceryl-10 caprylate/caprate.

Typically, at least one or more polyglyceryl esters may be present in an amount between 1 w/w %-80 w/w %, based on the total weight of the composition. Suitably, at least one or more polyglyceryl esters may be present in an amount between 1 w/w %-30 w/w %, based on the total weight of the composition. Desirably, at least one or more polyglyceryl esters may be present in an amount between 5 w/w %-25 w/w %, based on the total weight of the composition. For example, one or more polyglyceryl esters can be present from the composition in an amount generally greater than about 1% by weight, such as greater than about 2% by weight, such as greater than about 3% by weight, such as greater than about 4% by weight, such as greater than about 5% by weight, such as greater than about 6% by weight, such as greater than about 7% by weight, such as greater than about 8% by weight and generally less than about 40% by weight, such as less than about 35% by weight, such as less than about 30% by weight, such as less than about 25% by weight, such as less than about 20% by weight, such as less than about 15% by weight.

The composition of the present disclosure may optionally contain one or more anionic surfactants. In one aspect, for instance, the composition contains very low amounts of anionic surfactants. Typical anionic surfactants for use in the present invention include salts of C₁₂ to C₁₈ carboxylic acids, ethoxylated carboxylic acids, ester carboxylates and ethoxylated ester carboxylates and sarcosinates. Other suitable anionic surfactants include sulfates and sulfonates, for example alkyl sulfates, alkyl ether sulfates, alcohol sulfates, alcohol ether sulfates, α-olefin sulfonates, linear alkyl sulfonates; and phosphate esters.

Suitable anionic surfactants include, but are not limited to, fatty acids; alkali metal salts of mono- or dialkyl sulfates; mono- or dialkyl ether sulfates; lauryl ether sulfates; alkyl sulfonates; alkyl aryl sulfonates; primary alkane disulfonates; alkene sulfonates; hydroxyalkane sulfonates; alkyl glyceryl ether sulfonates; alpha-olefinsulfonates; alkyl phosphates; sulfonates of alkylphenolpolyglycol ethers; salts of alkyl sulfopolycarboxylic acid esters; alkyl sulfosuccinates and salts thereof, alkyl ether sulfosuccinates and salts thereof, non-acylated alkyl isethionates; fatty acid taurates; acyl taurates; products of condensation of fatty acids with oxy- and am inoalkanesulfonic acids; sulfated derivatives of fatty acids and polyglycols; alkyl and acyl sarcosinates; sulfoacetates; alkyl phosphates; alkyl phosphate esters; acyl lactates; alkanolamides of sulfated fatty acids and salts of lipoamino acids. Particularly exemplary salts of the above, where applicable, are the sodium, potassium, ammonium, magnesium and triethanolamine salts.

Examples additional anionic detersive surfactants include, but are not limited to, alkyl glyceryl ether sulfonate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Exemplary examples of anionic surfactants comprises one or more of a sodium lauroyl methyl isethionate, sodium cocoyl methyl isethionate, sodium oleoyl methyl isethionate, olefin sulphonates, sodium lauryl sulfate, olefin sulphonate, disodium laureth sulfosuccinate, sodium lauryl sulfoacetate, sulfonated methyl esters, sodium cocoyl glutamate, or sodium lauroyl sarcosinate, and the like.

Typically, at least one or more anionic surfactants, when present may be present in an amount between up to 80 w/w %; based on the total weight the solid bar shampoo composition. Suitably, at least one or more anionic surfactants may be present in an amount between 20 w/w %-70 w/w %; based on the total weight the solid bar shampoo composition. Desirably, at least one or more anionic surfactants may be present in an amount between 30 w/w %-60 w/w %; based on the total weight the solid bar shampoo composition. In another aspect, however, the composition is substantially free of anionic surfactants, particularly sulfate and/or sulfonate surfactants.

Examples of nonionic surfactants, include but are not limited to, reaction products of compounds having a hydrophobic group and a reactive hydrogen atom, for example aliphatic alcohols, acids, amides or alkyl phenols with alkylene oxides, especially ethylene oxide either alone or with propylene oxide (for example alkyl (C₆-C₂₂) phenol-ethylene oxide condensates, the condensation products of aliphatic (C₈-C₁₅) primary or secondary linear or branched alcohols with ethylene oxide, and products made by condensation of ethylene oxide with the reaction products of propylene oxide and ethylenediamine); long chain tertiary amine oxides, long chain tertiary phosphine oxides and dialkyl sulfoxides; alkyl amine oxides, alkyl amido amine oxides; alkyl tertiary phosphine oxides; alkoxyl alkyl amines; sorbitan; sorbitan esters; sorbitan ester alkoxylates; glycerol ester alkoxylates; sucrose esters; sugar amides, such as a polysaccharide amide; lactobionamides; and alkyl polysaccharide nonionic surfactants, for example alkylpolyglycosides. Typically, non-ionic surfactants are sucroglycerides and ethoxylated fatty alcohols, especially those derived from lauryl, cetylstearyl, stearyl, cetyl and oleocetyl alcohols, and the like.

Suitable non-ionic surfactants include, but are not limited to, alcohol ethoxylates and ethylene oxide/propylene oxide copolymer derived surfactants, sugar esters, especially sorbitan esters, alkyl polyglucosides, fatty acid ethoxylates or polyethylene glycol esters and partial esters, alkanolamides and amineoxides, fatty acid alkanolamides, ethylene glycol stearate and ethylene glycol distearate, glyceryl stearate (and) peg-100 stearate, and the like.

Typically, at least one or more nonionic surfactants, when present, may be present in in an amount between 1 w/w %-50 w/w %; based on the total weight of the surfactants in the solid bar shampoo composition. Suitably, at least one or more nonionic surfactants may be present in in an amount between 10 w/w %-40 w/w %; based on the total weight of the surfactants in the solid bar shampoo composition. Desirably, at least one or more nonionic surfactants may be present in in an amount between 20 w/w %-40 w/w %; based on the total weight of the surfactants in the solid bar shampoo composition.

Suitable cationic surfactants, include but are not limited to, fatty amine derivates or phosphonium quaternary ions, and quaternary ammonium compounds, tertiary amine salts, mono alkyl trimethyl ammonium chloride, mono alkyl trimethyl ammonium methyl sulfate, dialkyl dimethyl ammonium chloride, dialkyl dimethyl ammonium methyl sulfate, trialkyl methyl ammonium chloride and trialkyl methyl ammonium methyl sulfate, C₁₆-C₂₂ monoalkyl quaternary ammonium compounds, or trimethyl quaternary compounds, salts of primary, secondary, and tertiary fatty amines are also suitable cationic surfactants. The alkyl groups of such amines preferably have from C₁₂-C₂₂ atoms, and can be substituted or unsubstituted; and the like.

Examples of cationic surfactants, include but are not limited to, cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by halogen, (e.g., bromide), acetate, citrate, lactate, glycolate, phosphate nitrate, sulfate, or alkylsulfate, dimethylstearamine, dimethylsoyamine, soyamine, myristylamine, tridecylamine, ethylstearylamine, Ntallowpropane diamine, ethoxylated (with 5 moles of ethylene oxide) stearylamine, dihydroxyethylstearylamine, and arachidyl behenylamine, stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide; and the like.

Typically, at least one or more cationic surfactants, when present, may be present in an amount between 0.01 w/w %-25 w/w %; based on the total weight of the solid bar shampoo composition. Suitably, the solid bar shampoo comprises at least one or more cationic surfactant may be present in an amount between 0.01 w/w %-15 w/w %; based on the total weight of the solid bar shampoo composition. Desirably, the solid bar shampoo comprises at least one or more cationic surfactant may be present in an amount between 0.01 w/w %-10 w/w %; based on the total weight of the solid bar shampoo composition.

Examples of amphoteric surfactants, include but are not limited to, amphoteric or zwitterionic surfactants may be selected from betaines, for example alkyl betaines, alkylamidopropyl betaines, alkylamidopropyl hydroxy sultaines, alkylamphoacetates, alkylamphodiacetates, alkylamphopropionates, alkylamphodipropionates, alkyliminodipropionates, alkyliminodiacetate, or combinations thereof, and the like.

Examples of suitable acetate-based surfactants include lauroamphoacetate; alkyl amphoacetate; cocoampho(di)acetate; cocoamphoacetate; disodium cocoamphodiacetate; sodium cocoamphoacetate; disodium cocoamphodiacetate; disodium capryloamphodiacete; disodium lauroamphoacetate; sodium lauroamphoacetate and disodium wheatgermamphodiacetate, and the like.

Typically, at least one or more amphoteric surfactants, when present may be present in an amount between 1 w/w %-25 w/w %; based on the total weight of the solid bar shampoo composition. Suitably, the solid bar shampoo comprises at least one or more amphoteric surfactant may be present in an amount between 1 w/w %-15 w/w %; based on the total weight of the solid bar shampoo composition. Desirably, the solid bar shampoo comprises at least one or more amphoteric surfactant may be present in an amount between 1 w/w %-10 w/w %; based on the total weight of the solid bar shampoo composition.

Suitably, the present invention provides a solid bar shampoo composition, whereby the composition contains a low level of sulfate and/or sulfonate based surfactants, as is shown below in example 1. Desirably, the present invention provides a solid bar shampoo composition, whereby the composition is essentially free of sulfate and/or sulfonate based surfactants.

In another aspect, the present invention may further comprise one or more ingredients selected from fragrances, dyes, structuring aids, fillers, pH adjustment agents, chelating agents, antidandruff agents, biocidal agent, conditioning agents, essential oils, binding agent, natural extracts, botanical extracts, organic acids, alcohols, emulsifiers, natural plant material, fats, oils, essential fatty acids, preservatives, antioxidants, moisturizing agents, or coloring agents, and the like.

The fatty alcohol present in the concentrate composition may comprise, for instance, any suitable fatty alcohol having a carbon chain length from about C₄-C₃₆ carbon atoms, such as from about C₆-C₂₈ carbon atoms.

Suitably, the fatty alcohol may comprise a mixture of branched, unbranched, saturated or unsaturated fatty alcohols. Examples of fatty alcohols include, but are not limited to, capryl alcohol, pelargonic alcohol, capric alcohol, lauryl alcohol, myristyl alcohol, cetyl alcohol, palmitoleyl alcohol, stearyl alcohol, oleyl alcohol, arachidyl alcohol, behenyl alcohol, erucyl alcohol, lignoceryl alcohol, ceryl alcohol, mixtures thereof and the like.

Examples of preservatives include but are not limited to, personal care or cosmetic preservatives of one or more preservative of benzoic acid, sodium benzoate, benzyl alcohol, sorbic acid or salts thereof (i.e. potassium-sorbate), parabens, dehydroacetic acid, bronopol, triclosan, imidazolidinyl urea, PHMB, phenoxyethanol, DMDMH, ethylhexylglycerol, isothiazolones (such as CMIT/MIT, MIT); chlorhexidine, diazolidinyl urea, chlorphenesin, sodium hydroxymethylglycinate, benzethonoium chloride, IPBC (iodopropynyl butylcarbamate), salicylic acid, natural acids (such as citric, cinnamic, tartaric, malic, oxalic, lactic, ascorbic acids), combinations thereof and the like).

Examples of essential oils include, but are not limited to, balsam oil, pine oil, citrus oil, lime oil, jasmine oil, rose oil, muscatel sage oil, chamomile oil, lavender oil, clove oil, melissa oil, marula oil, coconut oil, mint oil, cinnamon leaf oil, lime blossom oil, Wa cholderbeerbl, vetiver oil, olibanum oil, galbanum oil and labdanum oil orange blossom oil, neroli oil, orange peel oil, sandalwood oil, anise oil, aniseed oil, arnica blossom oil, basil oil, bay oil, Chamberlain pacablütenöl, silver fir oil, noble fir cone oil, elemi oil, eucalyptus oil, fennel oil, pine needle oil, galbanum oil, geranium oil, ginger grass oil, Guaj akholzöl, Gurj unbalsamöl, helichrysum oil, Ho oil, ginger oil, iris oil, cajeput oil, calamus oil, camomile oil, camphor oil, Kanagaöl, cardamom oil, cassia oil, pine needle oil, Kopalvabalsamöl, coriander oil, spearmint oil, caraway oil, Kuminöl, lemongrass oil, ambrette seed oil, myrrh oil, mustard seed oil, clove oil, neroli oil, niaouli oil, olibanum oil, oregno oil, palmarosa oil, patchouli oil, petitgrain oil, pepper oil, peppermint oil, pimento oil, pine oil, palm oil, rose oil, rosemary oil, sandalwood oil, celery oil, sternanis oil, thuja oil, thyme oil, verbena, vetiver oil, wormwood oil, wintergreen oil, ylang-ylang oil, hyssop oil, cinnamon oil, cinnamon leaf oil, and cypress oil, theobroma oil (cocoa butter), or mixtures thereof, and the like.

Typically, the solid bar shampoo composition comprises 1 w/w %-80 w/w % of one or more polyglyceryl esters, up to 80 w/w % of one or more anionic surfactants, 1 w/w %-25 w/w % of one or more amphoteric surfactants, 1 w/w %-50 w/w % of one or more nonionic surfactants, 0.01 w/w %-20 w/w % essential oils, 0.1 w/w %-10 w/w % fatty alcohol, 0.1 w/w %-10 w/w % fatty acid, 0.01 w/w %-5 w/w % fragrances, or 0.01 w/w %-5 w/w % dyes.

Suitably, the solid bar shampoo composition comprises 1 w/w %-30 w/w % of one or more polyglyceryl esters, 30 w/w %-70 w/w % of one or more anionic surfactants, 1 w/w %-15 w/w % of one or more amphoteric surfactants, 10 w/w %-40 w/w % of one or more nonionic surfactants, 0.01 w/w %-20 w/w % essential oils, 0.1 w/w %-10 w/w % fatty alcohol, 0.1 w/w %-10 w/w % fatty acid, 0.01 w/w %-2 w/w % fragrances, or 0.01 w/w %-2 w/w % dyes.

If desired, the solid shampoo composition may contain at least one cationic surfactant, wherein the composition comprises 0.01 w/w %-25 w/w % of one or more cationic surfactants, 1 w/w %-80 w/w % of one or more polyglyceryl esters, up to 80 w/w % of one or more anionic surfactants, 1 w/w %-25 w/w % of one or more amphoteric surfactants, 1 w/w %-50 w/w % of one or more nonionic surfactants, 0.01 w/w %-20 w/w % essential oils, 0.1 w/w %-10 w/w % fatty alcohol, 0.1 w/w %-10 w/w % fatty acid, 0.01 w/w %-5 w/w % fragrances, or 0.01 w/w %-5 w/w % dyes.

A further aspect of the present invention provides a solid bar shampoo composition, comprising (a) at least 60% of one or more surfactants, wherein one or more surfactant comprises a polyglyceryl ester and (b) at least one or more antidandruff agent.

Additionally, this new composition may further comprise one at least one or more surfactants whereby at least one or more is an anionic surfactants, cationic surfactants, nonionic surfactants or amphoteric surfactants; as described in the present invention.

An antidandruff agent herein refers to any substance, in particular, in the cosmetic treatment of dandruff conditions associated with the excessive proliferation of yeasts of the Malassezia genus on the scalp; or substances known to have fungicidal properties.

Examples of antidandruff agents include, but are not limited to spiculisporic acid and or salts thereof; essential oils, natural extracts, zinc pyrithione, piroctone olamine or seleni-um disulfide, acetylsalicylic acid, hydroxypyridinone or derivatives thereof [such as 2(1H)-Pyridinone, 1-hydroxy-6-(octyloxy)-; 2(1H)-Pyridinone, 1-hydroxy-6-(octyloxy)-, compd. with 2-aminoethanol (1:1)]; hydroxypyrimidinone derivatives, octopirox (piroctone olamine), pyrithione derivatives or salts thereof (such as calcium, magnesium, strontium, barium, zinc, cadmium, tin and zirconium salts); bispyrithione, octenidine or salts thereof; azole compounds (such as climbazole, ketoconazole, clotrimazole, econazole; isoconazole and miconazole), selenium or salts thereof, salicylic acid or salts thereof, or tea tree oil; antifungal polymers (such as amphotericin B or nystatin; ellagic acid, ethers thereof, ellagic acid salts and salts of its ethers (such as alkali metal or alkaline-earth metal salts, especially the sodium, potassium, calcium or magnesium salts)); sulfur in its various forms, cadmium sulfide, allantoin, coal or wood tars and derivatives thereof; undecylenic add, fumaric acid; and allylamines such as terbinafine; antifungal peptides (such as syringomycins, syringostatins, syringotoxins; nikkomycins, echinocandins, pneumocadins, aculeacins; mulundocadins, cecropins, α-defensins, β-defensins, novispirins, and mixtures thereof); or combinations thereof, and the like.

Suitable antidandruff agent include, but are not limited to, at least one or more of hydroxypyridinones or derivatives thereof; octopirox, pyrithione derivatives or salts thereof, bispyrithione, octenidine or salts thereof, ketoconazole, climbazole, selenium or salts thereof, salicylic acid or salts thereof, or tea tree oil; and the like.

Typically, at least one or more antidandruff agent may be present in an amount between 0.01 w/w %-10 w/w % antidandruff agent; based on the total weight of the composition. Suitably, at least one or more antidandruff agent may be present in an amount between 0.01 w/w %-5 w/w % antidandruff agent; based on the total weight of the composition. Desirably, at least one or more antidandruff agent may be present in an amount between 0.01 w/w %-2 w/w % antidandruff agent; based on the total weight of the composition.

Typically, the solid bar shampoo composition comprises 1 w/w %-80 w/w % of one or more polyglyceryl esters, up to 80 w/w % of one or more anionic surfactants, 1 w/w %-25 w/w % of one or more amphoteric surfactants, 1 w/w %-50 w/w % of one or more nonionic surfactants, 0.01 w/w %-20 w/w % essential oils, 0.1 w/w %-10 w/w % fatty alcohol, 0.1 w/w %-10 w/w % fatty acid, 0.01 w/w %-10 w/w % antidandruff agent, 0.01 w/w %-5 w/w % fragrances, 0.01 w/w %-2 w/w % moisturizing agent, or 0.01 w/w %-5 w/w % dyes.

Suitably, the solid bar shampoo composition comprises 1 w/w %-30 w/w % of one or more polyglyceryl esters, 30 w/w %-70 w/w % of one or more anionic surfactants, 1 w/w %-15 w/w % of one or more amphoteric surfactants, 10 w/w %-40 w/w % of one or more nonionic surfactants, 0.01 w/w %-20 w/w % essential oils, 0.1 w/w %-7 w/w % fatty alcohol, 0.1 w/w %-7 w/w % fatty acid, 0.01 w/w %-5 w/w % antidandruff agent, 0.01 w/w %-2 w/w % fragrances, 0.01 w/w %-2 w/w % moisturizing agent, or 0.01 w/w %-2 w/w % dyes.

Alternately, at one or more cationic surfactant when added, provides a solid bar composition comprising 1 w/w %-80 w/w % of one or more polyglyceryl esters, up to 80 w/w % of one or more anionic surfactants, 1 w/w %-25 w/w % of one or more amphoteric surfactants, 1 w/w %-50 w/w % of one or more nonionic surfactants, 0.01 w/w %-25 w/w % of one or more cationic surfactants, 0.01 w/w %-20 w/w % essential oils, 0.1 w/w %-10 w/w % fatty alcohol, 0.1 w/w %-10 w/w % fatty acid, 0.01 w/w %-10 w/w % antidandruff agent, 0.01 w/w %-5 w/w % fragrances, 0.01 w/w %-2 w/w % moisturizing agent, or 0.01 w/w %-5 w/w % dyes.

The following examples illustrate the invention without limitation. All parts and percentages are given by weight unless otherwise indicated.

It will be understood that each of the elements described in the examples below, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims.

The foregoing embodiments are presented by way of example only; the scope of the present disclosure is to be limited only by the following claims.

Sample Preparation for Examples 1-4.

Compositions for the examples below were prepared by melting Phase A ingredients together with light stirring at 70° C.; then mixing Phase B ingredients to form a homogenous mixture. Lastly Phase C was added and the mixture was stirred for 5 minutes or until homogenous and pour into molds.

### Example 1. Low Sodium Lauryl Sulfate Solid Shampoo Bar

TABLE 1

| Phase | Raw material | Sample 1 (w/w %) |
|---|---|---|
| A | Isethionate | 30 |
| A | Disodium Cocoamphodiacetate | 10 |
| A | polyglyceryl-10 laurate | 10 |
| A | glyceryl stearate (and) peg-100 stearate | 3 |
| A | Sodium lauryl sulfate | 25 |
| B | theobroma oil | 5 |
| B | Marula oil | 1 |
| B | stearalkonium chloride | 8 |
| B | Stearic acid | 3 |
| B | Cetyl alcohol | 4 |
| C | Dye | 0.01 |
| C | fragrance | 1 |

### Example 2. Solid Shampoo Bar 1 (Sodium Lauryl Sulfate Free)

TABLE 2

| Phase | Raw material | Sample 1 (w/w %) |
|---|---|---|
| A | Isethionate | 40 |
| A | Disodium Cocoamphodiacetate | 10 |
| A | polyglyceryl-10 laurate | 20 |
| A | glyceryl stearate (and) peg-100 stearate | 6 |
| B | theobroma oil | 5 |
| B | Marula oil | 1 |
| B | stearalkonium chloride | 7 |
| B | Stearic acid | 6 |
| B | Cetyl alcohol | 4 |
| C | Dye | 0.01 |
| C | fragrance | 1 |

### Example 3. Solid Shampoo Bar 2 (Sodium Lauryl Sulfate Free)

TABLE 3

| Phase | Raw material | Sample 1 (w/w %) |
|---|---|---|
| A | Isethionate | 30 |
| A | Olefin sulphonate | 20 |
| A | Disodium Cocoamphodiacetate | 5 |
| A | polyglyceryl-10 laurate | 15 |
| A | glyceryl stearate (and) peg-100 stearate | 6 |
| B | theobroma oil | 5 |
| B | Marula oil | 1 |
| B | stearalkonium chloride | 7 |
| B | Stearic acid | 6 |
| B | Cetyl alcohol | 4 |
| C | Dye | 0.01 |
| C | fragrance | 1 |

### Example 4. Solid Shampoo Bar 3 (Sodium Lauryl Sulfate Free)

TABLE 4

| Phase | Raw material | Sample 1 (w/w %) |
|---|---|---|
| A | Isethionate | 30 |
| A | Olefin sulphonate | 20 |
| A | Disodium Cocoamphodiacetate | 5 |
| A | polyglyceryl-10 laurate | 15 |
| A | Lactobacillus/Milk Solids/Soybean Oil | 1 |
| | Ferment & Cyclodextrin | |
| A | glyceryl stearate (and) peg-100 stearate | 7 |
| A | Stearic acid | 7 |
| B | theobroma oil | 5 |
| B | Marula oil | 1 |
| B | stearalkonium chloride | 7 |
| B | Isododecane & PPG-3 Myristyl Ether | 1 |
| | Neoheptanoate | |
| C | Dye | 0.01 |
| C | fragrance | 1 |

While the invention has been described above with references to specific embodiments thereof, it is apparent that many changes, modifications and variations can be made without departing from the invention concept disclosed herein. Accordingly, it is intended to embrace all such changes, modifications, and variations that fall within the spirit and broad scope of the appended claims.

What is claimed is:

1. A solid bar shampoo composition comprising at least 60 w/w % of one or more surfactants, based on the total weight of the composition, wherein at least one of the surfactants comprises a polyglyceryl ester.

2. The solid bar shampoo composition, according to claim 1, wherein at least one or more surfactants further comprises at least one or more anionic surfactants, cationic surfactants, nonionic surfactants or amphoteric surfactants.

3. The solid bar shampoo composition, according to claim 1, comprising 60 w/w %-99 w/w % of one or more surfactants, based on the total weight of the composition.

4. The solid bar shampoo composition, according to claim 3, comprising 70 w/w %-85 w/w % of one or more surfactants, based on the total weight of the composition.

5. The solid bar shampoo composition, according to claim 1, wherein the polyglyceryl fatty ester is derived from (a) a polyglycerol component built up from 2 to 12 molecules of glycerol, based on an average, and (b) a fatty acid comprises a caprylic acid, capric acid, lauric acid, myristic acid, palmitic acid, stearic acid, arachidic acid, behenic acid, lignoceric acid, cerotic acid, oleic acid, or decaoleic acid.

6. The solid bar shampoo composition, according to claim 5, wherein the polyglyceryl fatty ester comprises one or more of a polyglyceryl-10 laurate, polyglyceryl-10 decaoleate; polyglyceryl-3 monostearate; polyglyceryl-6 distearate, polyglyceryl-10 stearate; polyglyceryl-10 oleate; polyglyceryl-10 dipalmitate, or polyglyceryl-10 caprylate/caprate.

7. The solid bar shampoo composition, according to claim 1, wherein one or more polyglyceryl esters is present in an amount between 1 w/w %-80 w/w %, based on the total weight of the composition.

8. The solid bar shampoo composition according to claim 2, wherein the anionic surfactant comprises one or more of a sodium lauroyl methyl isethionate, sodium cocoyl methyl isethionate, sodium oleoyl methyl isethionate, olefin sulphonates, sodium lauryl sulfate, olefin sulphonate, disodium laureth sulfosuccinate, sodium lauryl sulfoacetate, sulfonated methyl esters, sodium cocoyl glutamate, or sodium lauroyl sarcosinate.

9. The solid bar shampoo composition according to claim 8, wherein the anionic surfactant is present in an amount up to 80 w/w %; based on the total weight of the solid bar shampoo composition.

10. The solid bar shampoo composition, according to claim 2, wherein the amphoteric surfactant comprises disodium cocoamphodiacetate.

11. The solid bar shampoo composition according to claim 10, wherein the amphoteric surfactant is present in an amount between 1 w/w %-25 w/w %; based on the total weight of the solid bar shampoo composition.

12. The solid bar shampoo composition, according to claim 2, wherein the nonionic surfactant comprises glyceryl stearate, peg-100 stearate or a mixture thereof.

13. The solid bar shampoo composition according to claim 12, wherein the nonionic surfactant is present in an amount between 1 w/w %-50 w/w %; based on the total weight of the solid bar shampoo composition.

14. The solid bar shampoo, according to claim 1, wherein the composition is essentially free of sulfate and/or sulfonate based surfactants.

15. The solid bar shampoo composition, according to claim 2, wherein the cationic surfactant is present in an amount between 0.01 w/w %-25 w/w %; based on the total weight of the composition.

16. The solid bar shampoo, according to claim 1, wherein the composition is a non-saponified bar shampoo.

17. The solid bar shampoo composition according to claim 1, further comprising one or more ingredients selected from fragrances, dyes, structuring aids, fillers, pH adjustment agents, chelating agents, antidandruff agents, biocidal agent, conditioning agents, essential oils, binding agent, natural extracts, botanical extracts, organic acids, alcohols, emulsifiers, natural plant material, fats, oils, essential fatty acids, preservatives, antioxidants, moisturizing agents, or coloring agents.

18. The solid bar shampoo composition, according to claim 1, comprising 1 w/w %-80 w/w % of one or more polyglyceryl esters, up to 80 w/w % of one or more anionic surfactants, 1 w/w %-25 w/w % of one or more amphoteric surfactants, 1 w/w %-50 w/w % of one or more nonionic surfactants, 0.01 w/w %-25 w/w % of one or more cationic surfactants; 0.01 w/w %-20 w/w % essential oils, 0.1 w/w %-10 w/w % fatty alcohol, 0.1 w/w %-10 w/w % fatty acid, 0.01 w/w %-5 w/w % fragrances, or 0.01 w/w %-5 w/w % dyes.

19. A solid bar shampoo composition, comprising (a) at least 60% of one or more surfactants, wherein at least one of the surfactants comprises a polyglyceryl ester and (b) at least one or more antidandruff agent.

20. The solid bar shampoo composition, according to claim 19, wherein the one at least one or more surfactants further comprises of at least one or more of anionic surfactants, cationic surfactants, nonionic surfactants or amphoteric surfactants.

21. The solid shampoo bar composition, according to claim 19, wherein the antidandruff agent comprises at least one or more of hydroxypyridinones or derivatives thereof, octopirox, pyrithione derivatives or salts thereof, bispyrithione, octenidine or salts thereof, ketoconazole, climbazole, selenium or salts thereof, salicylic acid or salts thereof, or tea tree oil.

22. The solid bar shampoo composition, according to claim 19, comprising 1 w/w %-80 w/w % of one or more polyglyceryl esters, up to 80 w/w % of one or more anionic surfactants, 1 w/w %-25 w/w % of one or more amphoteric surfactants, 1 w/w %-50 w/w % of one or more nonionic surfactants, 0.01 w/w %-25 w/w % of one or more cationic surfacants; 0.01 w/w %-20 w/w % essential oils, 0.1 w/w %-10 w/w % fatty alcohol, 0.1 w/w %-10 w/w % fatty acid, 0.01 w/w %-10 w/w % antidandruff agent, 0.01 w/w %-5 w/w % fragrances, 0.01 w/w %-2 w/w % moisturizing agent, or 0.01 w/w %-5 w/w % dyes.
